(19) United States
(12) Patent Application Publication
(10) Pub. No.: US 2015/0224348 A1
(43) Pub. Date: Aug. 13, 2015

(54) METHOD AND APPARATUS FOR SELECTIVE NERVE STIMULATION

(71) Applicant: Cardiac Pacemakers, Inc., St. Paul, MN (US)

(72) Inventors: Anand Iyer, Summerville, SC (US); Rodney W. Salo, Fridley, MN (US); Anthony V. Caparso, San Jose, CA (US); Paul A. Haefner, Circle Pines, MN (US)

(21) Appl. No.: 14/694,462

(22) Filed: Apr. 23, 2015

Related U.S. Application Data
(63) Continuation of application No. 11/276,066, filed on Feb. 13, 2006, now abandoned.

(51) Int. Cl. A61N 7/00; A61N 1/362

(52) U.S. Cl. CPC A61N 7/00; A61N 1/362

(57) ABSTRACT

Various aspects relate to a device. Various device embodiments include at least a first and a second transducer, and a controller. The first transducer is adapted to be positioned to direct a first energy wave toward a neural target, and the second transducer is adapted to be positioned to direct a second energy wave toward the neural target. The controller is connected to the transducers to generate the first energy wave with a first predetermined phase and a first predetermined amplitude from the first transducer and to generate the second energy wave with a second predetermined phase and a second predetermined amplitude from the second transducer. The amplitudes are selected so that a neural stimulation threshold is reached only during constructive wave interference. The phases are selected so that the first and second energy waves constructively interfere at the neural target. Other aspects and embodiments are provided herein.

### CLAIM OF PRIORITY

This application is a continuation of U.S. application Ser. No. 11/276,066, filed Feb. 13, 2006, which is hereby incorporated by reference in its entirety.

### TECHNICAL FIELD

This application relates generally to medical devices and, more particularly, to devices and methods to stimulate nerves.

### BACKGROUND

Neural stimulation therapy has been proposed to treat a number of conditions, such as eating disorders, allergies, sexual dysfunction, pain, migraines, depression, steep disorders, movement disorders, epilepsy, and the like. Neural stimulation has also been proposed as, or as part of cardiac therapies, such as therapies to treat or control heart rhythms, to improve contractility and reverse remodel a heart, to reduce injury after a myocardial infraction, to treat hypertension, and the like.

It is desirable to be able to stimulate a specific nerve, or specific nerve fiber(s) within a nerve so as to obtain a desired neural stimulation effect while avoiding the stimulation of other proximate nerves and corresponding unintended neural stimulation effect(s).

### SUMMARY

Various aspects relate to a device. Various device embodiments include at least a first and a second transducer, and a controller. The first transducer is adapted to be positioned to direct a first energy wave toward a neural target, and the second transducer is adapted to be positioned to direct a second energy wave toward the neural target. The controller is connected to the transducers to generate the first energy wave with a first predetermined phase and a first predetermined amplitude from the first transducer and to generate the second energy wave with a second predetermined phase and a second predetermined amplitude from the second transducer. The amplitudes are selected so that a neural stimulation threshold is reached only during constructive wave interference. The phases are selected so that the first and second energy waves constructively interfere at the neural target. Other aspects and embodiments are provided herein.

Various aspects relate to a system. Various system embodiments comprise a plurality of ultrasound transducers and a controller. Each ultrasound transducer is adapted to be positioned to direct an ultrasound signal toward a neural target. The controller is adapted to deliver an electrical signal to each of the plurality of ultrasound transducers to generate the ultrasound signal toward the neural target. The controller is adapted to control a phase of the electrical signal to each of the plurality of ultrasound transducers to cause resulting ultrasound signals from the plurality of ultrasound transducers to constructively interfere at the neural target and provide sufficient energy to stimulate the neural target.

Various aspects relate to a method for stimulating a neural target. According to various method embodiments, a first energy wave is generated from a first position toward the neural target. The first energy wave has a first phase and has a first predetermined amplitude insufficient to stimulate the neural target by itself A second energy wave is generated from a second position toward the neural target. The second energy wave has a second phase and has a second predetermined amplitude insufficient to stimulate the neural target by itself The first phase of the first energy wave and the second phase of the second energy wave are selected to provide constructive interference at the neural target for use in delivering an energy capable of stimulating the neural target.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIGS. 1-3 illustrate waveforms in general, and are illustrative of interference for various waves, including acoustic (e.g. ultrasound), RF and infrared waves.

FIG. 4 illustrates an embodiment of an ultrasound transducer.

FIG. 5 illustrates an ultrasound transducer, and the near field and far field for an ultrasound transducer.

FIG. 6 illustrates an embodiment of multi-element transducer, according to various embodiments, which can be used to provide a steered or focused beam.

FIG. 7 illustrates a steered beam, according to various embodiments.

FIG. 8 illustrates a focused beam that can be generated, according to various embodiments.

FIGS. 9-11 illustrate neural target(s), a plan view of an imaginary axis through the neural target, and ultrasound transducers, according to various embodiments.

FIGS. 12-15 illustrate views of the neural target(s) along an imaginary axis through the neural target, and further illustrate ultrasound transducers about the neural target, according to various embodiments.

FIGS. 16A and 16B illustrate a nerve cuff around a nerve, according to various embodiments; and FIGS. 16C and 16D illustrate an intravascular device positioned proximate to a target nerve, according to various embodiments.

FIG. 17 illustrates an embodiment with external transducers.

FIGS. 18A-18B illustrate some device embodiments that provide selective nerve stimulation.

FIGS. 19A-19B illustrate some device embodiments that provide selective nerve stimulation and CRM therapy.

FIG. 20 illustrates an implantable medical device (IMD), according to various embodiments of the present subject matter.

FIG. 21 illustrates an implantable medical device (IMD) having a neural stimulation (NS) component and cardiac rhythm management (CRM) component, according to various embodiments of the present subject matter.

FIG. 22 illustrates an APM system according to various embodiments of t present subject matter.

FIG. 23 illustrates a method to selectively stimulate a desired neural target, according to various embodiments.

### DETAILED DESCRIPTION

The present subject matter directs two or more energy waveforms to a desired neural stimulation target. The energy from each waveform alone is not sufficient to stimulate the neural target, but the combination of energy waveforms at the neural stimulation target is greater than the neural stimulation threshold for the target. Examples of waveforms that may be used include acoustic waveforms such as ultrasound waveforms, as well as RF, microwave and light (e.g. infrared) waveforms. Ultrasound waveforms are described below. One of ordinary skill in the art will understand, upon reading and comprehending this disclosure, how to apply the teachings provided herein to focus other stimulation waveforms generated by corresponding transducers to a desired stimulation focal point. A brief overview of waveform interference is provided below.

### Waveform Interference

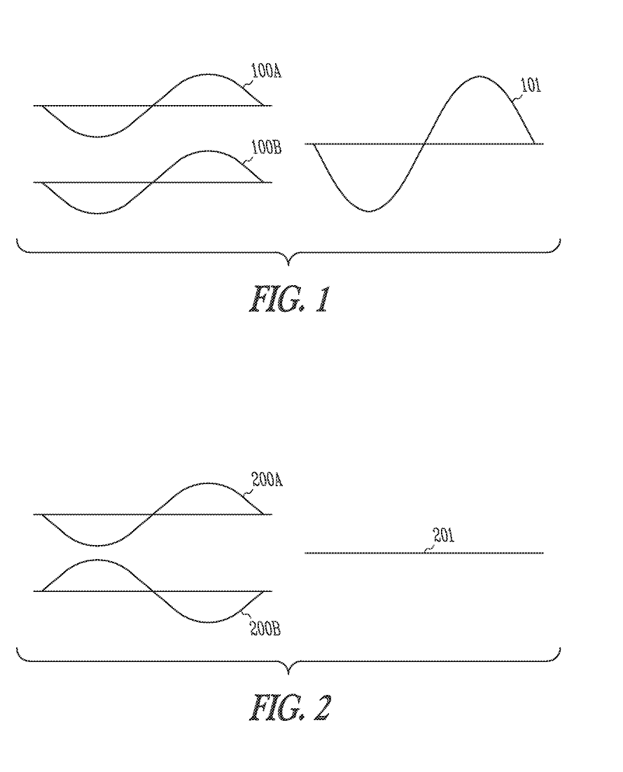

When two or more waves simultaneously and independently travel through the same medium at the same time, their effects are superpositioned and result in wave interferences. Constructive interference occurs when the wave amplitudes reinforce each other and results in a wave with a greater amplitude; and destructive interference occurs when the wave amplitudes oppose each other and results in waves of reduced amplitude. FIG. 1 provides a simple illustration of constructive interference by illustrating two identical waves 100A and 100B in phase with each, and the resulting superpositioned waveform 101 with twice the amplitude of either wave 100A or wave 100B. FIG. 2 provides a simple illustration of destructive interference by illustrating two identical waveforms 200A and 200B 180 degrees out of phase with respect to each other, and the resulting superpositioned waveform 201, which illustrates that wave 200A and wave 200B cancel each other. FIG. 3 illustrates constructive and destructive interference with more complex waveforms. The figure illustrates a first wave 300A and a second wave 300B, and a superimposed resulting waveform 301. The first and second waves destructively interfere with each other in regions 302, and constructively interfere with each other in regions 303. The present subject matter uses such constructive interference to deliver energy above a neural stimulation threshold for a desired neural target using individual wave energies significantly less than the neural stimulation threshold for the neural target.

### Ultrasound Stimulation

FIGS. 1-3 illustrate waveforms in general, and are illustrative of interference for various waves, including acoustic (e.g. ultrasound), RF and infrared waves. Sound is a pressure wave which consists of compressions and rarefactions. A compression tends to pull particles together into a small region of space, thus creating a high pressure region; and a rarefaction tends to push particles apart, thus creating a low pressure region. The interference of sound waves causes the particles of the medium to behave in a manner that reflects the net effect of the two individual waves upon the particles. For example, if a compression (high pressure) of one wave occurs with a compression (high pressure) of a second wave at the same location in the medium, then the waves constructively interfere and the net effect is that that particular location will experience a greater pressure. If two rarefactions (two low pressure disturbances) from two different sound waves occur at the same location, then the waves constructively interfere and the net effect is that that particular location will experience an even lower pressure. If two sound waves interfere at a given location in such a way that the compression of one wave meets up with the rarefaction of a second wave, the waves destructively interfere. The tendency of the compression to push particles together works against the tendency of the rarefactions to pull particles apart.

Nerves have been stimulated using ultrasound. It is believed that the ultrasound stimulation mechanically stimulates the neural structures through displacement of the medium. The ultrasound stimulation may also heat the tissue, which may also contribute to neural stimulation.

Some embodiments use at least two crystals to focus the energy, and some embodiments use at least three crystals to focus the energy. The energy from each crystal is not individually high enough to stimulate the nerve, but the combination of crystals is capable of stimulating the nerve when the energy wave from each constructively interfere.

Aspects of the present subject matter are directed to selective nerve stimulation. For example, the present subject matter provides stimulation waveforms toward the neural target using transducers located at various radial positions with respect to an imaginary axis that passes through the neural target. Positioning the transducers at radial positions with relatively wide angles, such as greater than or equal to 45 degrees, the energy waves are able to be focused with greater accuracy and selectivity. Additional selectivity can be achieved using three or more transducers radially positioned about the imaginary axis passing through a neural target. Each transducer produces a waveform with an energy, such that only a constructive interference of all waveforms at the focal point provides sufficient stimulation energy greater than a threshold to stimulate the neural target. The focal point of the energy beams can be adjusted to selectively stimulate parts of a nerve bundle. For example, the focal point can be changed by changing the phase of the energy, by physically adjusting the position or orientation of the crystals, or a combination of physically adjusting the orientation of the crystals or the phase of the energy.

Various neural stimulation waveforms can be used. In a square waveform, for example, a pulse width and amplitude can be adjusted to minimize stimulation of surrounding fiber populations, and a duty cycle can be varied to increase or decrease rate of stimulation. An appropriate feedback signal that reflects a desired or undesired response can be used to determine whether the energy has been focused on a desired nerve bundle.

Thus, the present subject matter can be used to stimulate different fibers within the same nerve bundle to produce individual effects. Aspects of the present subject matter have the potential to provide neural stimulation that is selective in the number of axons stimulated. Selective nerve stimulation can be achieved without penetrating the nerve, without relatively complex stimulation waveforms, and without steering currents.

Two or more ultrasonic crystals are spaced radially around a nerve bundle. For example, three crystals can be spaced 60 degrees apart from each other with respect to an imaginary axis passing through a neural target. In order to selectively stimulate a particular bundle of fibers within the nerve, the energy and timing of the electrical pulses to the crystals are adjusted to cause constructive interference of the propagated ultrasonic energy to bring it above the threshold necessary for stimulation at the site of interest within the nerve. The pulse width and pulse amplitude can be adjusted to minimize stimulation of surrounding fiber populations and the duty-cycle can be increased or decreased to alter the rate of stimulation. Different sized fibers within the same bundle (e.g. motor or sensory) can be stimulated selectively to create individual effects. Thus, for example, motor nerves could be stimulated to cause a hand to close and sensory fibers could be stimulated to generate a corresponding feeling of pressure. The present subject matter could also be used for vagal stimulation to control remodeling, reduce hypertension, improve wound healing, etc. as well as for stimulation of motor nerves and sensory nerves in cases of paralysis.

Since physical contact with the target nerve is not necessary, the transducers can be positioned using a nerve cuff to surround only the nerve, or can be positioned to surround a larger, more stable structure such as the nerve and an adjacent vessel, or can be externally positioned. Examples of externally-positioned transducers include transducers placed around a neck to stimulate a nerve such as a vagus nerve, or transducers placed around a limb to stimulate a corresponding nerve in the limb. Such transducers can be incorporated in collars, bracelets, or patches, for example, for use in stimulating the neck, arm or leg. A desired fiber can be stimulated, regardless of the specific geometry and makeup of the nerve.

The present subject matter can be used wherever nerve stimulation is desired, as it is selective and controllable without requiring direct contact with the nerve. The transfer of ultrasonic energy to the nerve is efficient, such that a relatively small battery can be used in implantable devices.

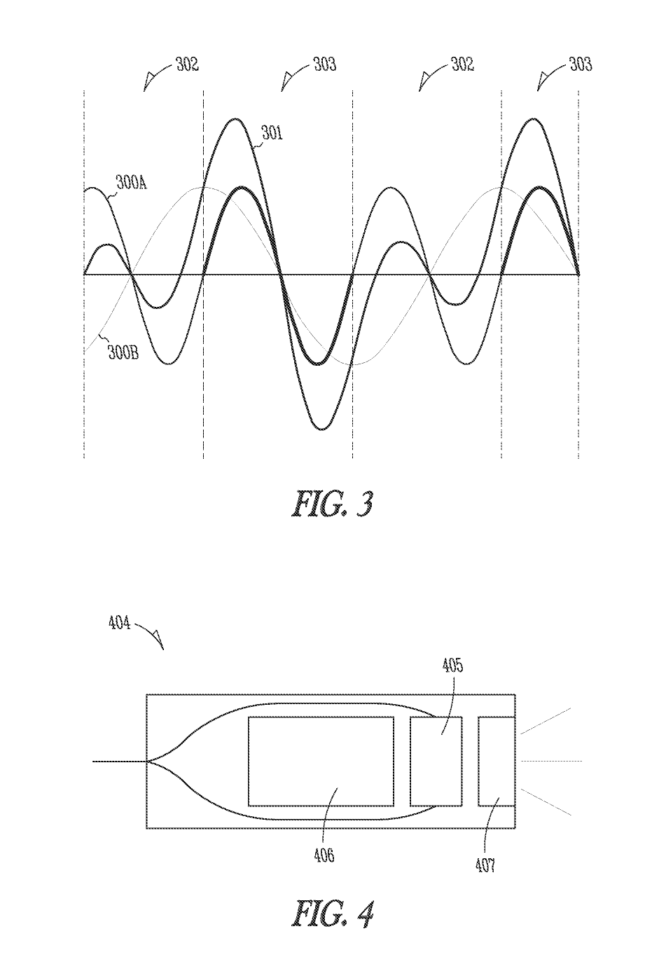

FIG. 4 illustrates an embodiment of an ultrasound transducer. Piezoelectric crystals, for example, can be used to focus ultrasound energy to an adjustable focal point. The illustrated transducer 404 includes a piezoelectric element 405 disposed between a backing material 406 and a matching layer 407. The piezoelectric element provides a mechanical movement in response to an electrical signal. Examples of piezoelectric elements include quartz crystal and polarized ferroelectrics, both of which have electric dipoles in their construction that realign under the presence of an applied voltage, causing the element to reshape. The matching layer mimics the properties of the tissue, to reduce or eliminate energy reflections, and the backing layer reduces vibration and echoes.

The transducer generates an ultrasound beam. The shape of an ultrasound beam depends on the radius and resonant frequency of the transducer. The ultrasound beam initially converges through a near field region, and diverges through a far field region.

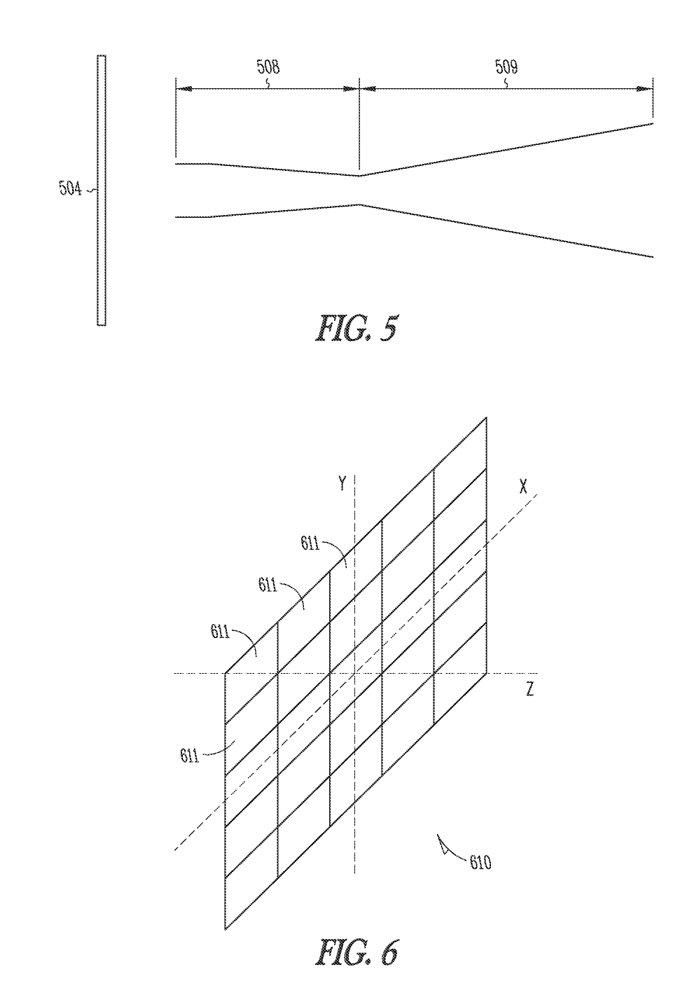

FIG. 5 illustrates an ultrasound transducer 504, and the near field 508 and far field 509 fir an ultrasound transducer. The near field length is a²/λ, where “a” represents the radius of the transducer face, and “λ” represents the wavelength. The frequency of the signal is related to wavelength, as represented by the expression f=v/λ, where f represents the frequency of the energy signal and v represents the velocity of the energy wave. The far field divergence angle is presented as θ=sin⁻¹((0.61*λ)/a).

The direction of the sound waves can be adjusted through the use of a multi-element transducer, and by adjusting the phase offset of different elements of the transducer. A steered beam can leave the transducer at an angle by having elements on one end have a phase that lead elements of the other end. A focused beam can be generated by having the phase of the outer elements lead the inner elements.

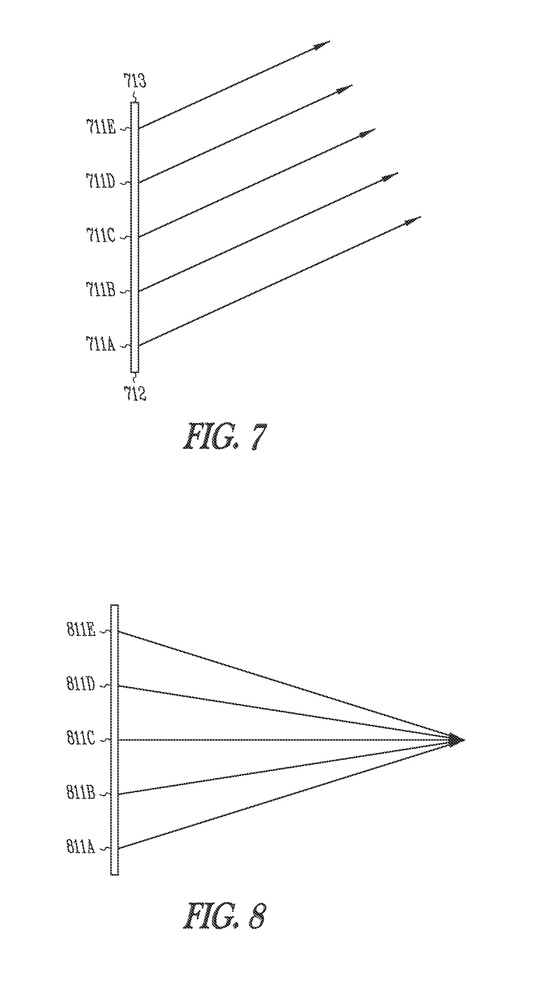

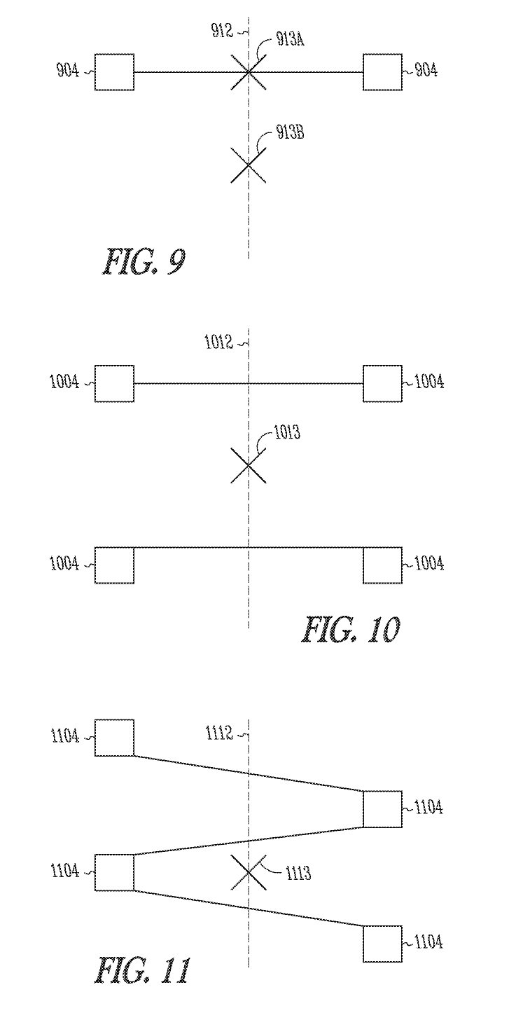

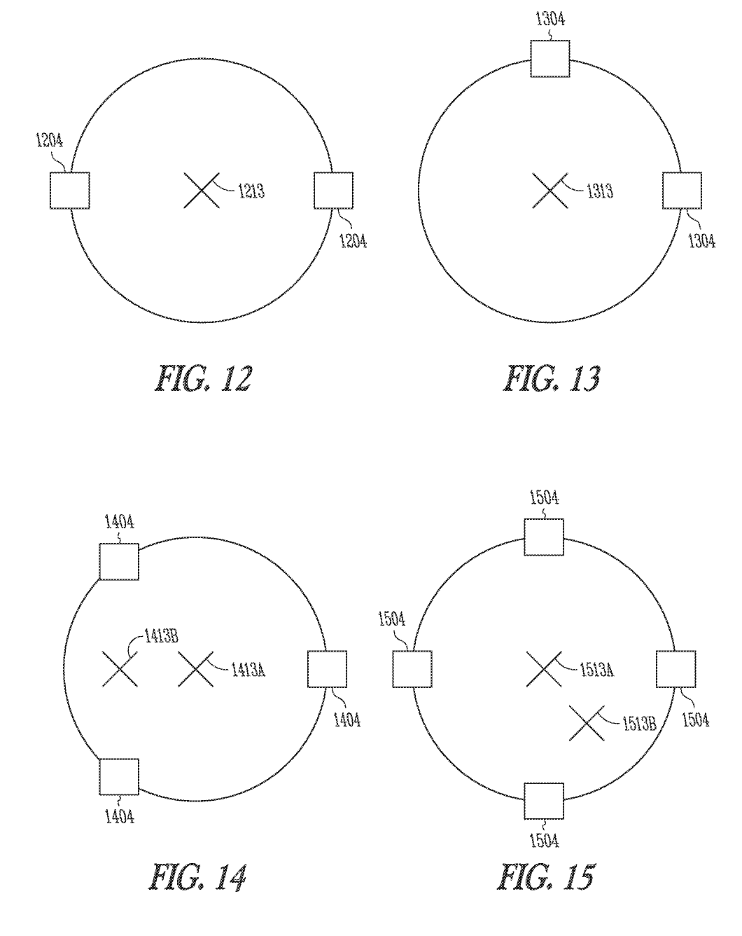

FIG. 6 illustrates an embodiment of multi-element transducer 610, according to various embodiments, which can be used to provide a steered or focused beam. Various multi-element transducers can be used. Each of the elements 611 can be individually controlled to provide a desired stimulation signal at a desired phase. FIG. 7 illustrates a steered beam, according to various embodiments. A steered beam can leave the transducer at an angle by having elements on one end 712 have a phase that leads elements of the other end 713. For example, the phase of element 711A can lead the phase of element 711B by a predetermined rotational angle, which can lead the phase of element 711C by the same rotational angle, which can lead the phase of element 711D by the same rotational angle, which can lead the phase of element 711E by the same rotational angle. FIG, 8 illustrates a focused beam that can be generated, according to various embodiments. For example, the phase of elements 811A and 811E can lead the phase of elements 811B and 811D by a predetermined rotational angle, which can lead the phase of element 811C by a predetermined rotational angle. The multi-element transducers illustrated in FIGS. 7 and 8 can be one-dimensional linear arrays, or two-dimensional arrays such as illustrated in FIG. 6. Those of ordinary skill in the art will understand, upon reading and comprehending this disclosure, how to control the phase of the elements in the linear arrays to steer or focus the beam within a plane that includes the linear array, and how to control the phase of the elements in the two-dimensional arrays to steer or focus the beam within a three-dimensional volume.

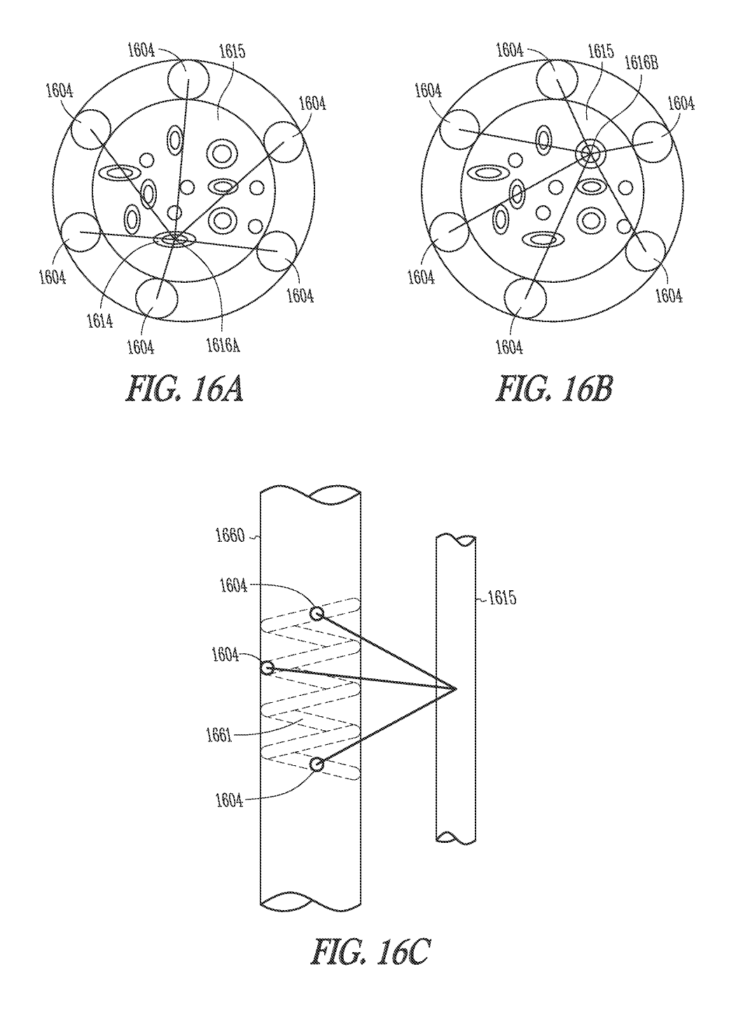

FIGS. 9-11 illustrate neural target(s), a plan view of an imaginary axis through the neural target, and ultrasound transducers, according to various embodiments. These figures illustrate that the transducers 904, 1004, 1104 can be positioned about an imaginary axis 912, 1012, 1112 that extends through a neural target 913A, 913B, 1013, 1113. The transducers do not need to be the same distance from the axis, nor do the neural targets need to be in the same plane as the transducers. Additionally, various transducers arrangements and orientations can be used. The transducers can be all in the same plane, such as illustrated in FIG. 9, or in different planes such as illustrates in FIGS. 10 and 11. The transducers can be controlled to stimulate any neural target along the illustrated imaginary axes or other locations not shown in the figures. Any of the transducers can be single-element or multi-element transducers.

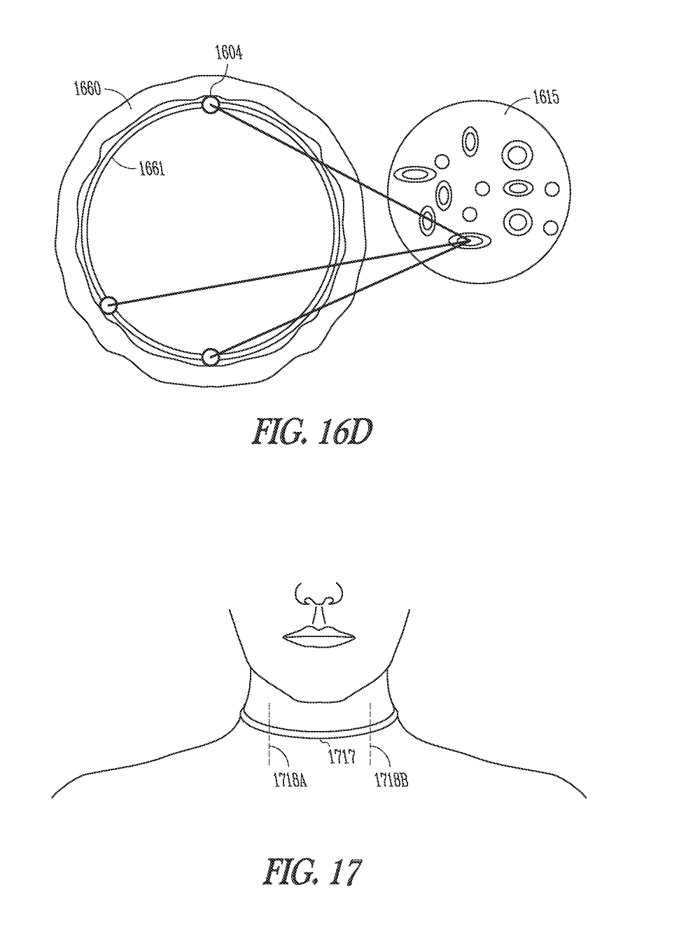

FIGS. 12-15 illustrate views of the neural target(s) along an imaginary axis through the neural target, an further illustrate ultrasound transducers about the neural target, according to various embodiments. The transducers 1204, 1304, 1404, 1504 can be controlled to create desired constructive interference at neural targets 1213, 1313, 1413A, 1414B, 1513A, 1513B. These neural targets are not limited to the positions illustrated in the figures.

Implantable Transducers (e.g. Nerve Cuffs)

Various embodiments provide implantable transducers. Some transducer embodiments can be positioned on leads. Some transducer embodiments can be implanted subcutaneously. Sonic transducer embodiments are implanted intravascularly. Some transducer embodiments include nerve cuff structures that include at least two transducers. Some transducer embodiments include nerve cuff structures that include three or more transducers. According to various embodiments, the transducers on the cuffs are oriented to direct/redirect stimulation energy to any area within the cuff. The cuffs can be constructed to circumscribe or at least partially circumscribe the nerve or the nerve and an adjacent stable structure such as an adjacent blood vessel. The term circumscribe is not intended to limit the cuff to a particular annular shape. In some embodiments, the transducers can be powered by and be in communication with an implantable device (e.g. can). Some transducer embodiments include power circuitry and communication circuitry for self-powering its own stimulation, and coordinating the stimulation with other transducers for a desired therapy.

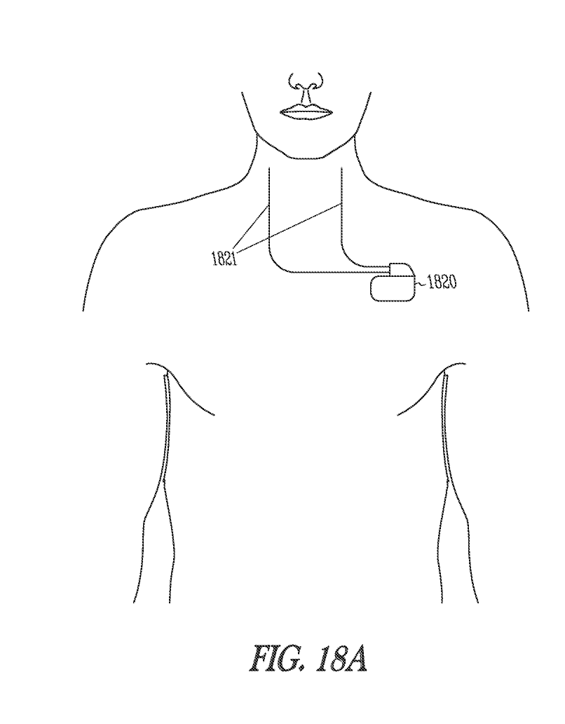

FIGS. 16A and 16B illustrate a nerve cuff 1614 around a nerve 1615, according to various embodiments. The illustrated nerve 1615 includes a number of nerve fiber bundles or fascicles, such as illustrated at 1616A and 1616B. The fascicles includes a number of axons that provide neural pathways. The transducers 1604 in the nerve cuffs 1615 can be controlled to cause the ultrasound energy to constructively interfere at various locations such as at 1616A and 1616B.

FIGS. 16C and 16D illustrate an intravascular device 1661 positioned proximate to a target nerve 1615, according to various embodiments. In the illustrated embodiment, the intravascular device 1661 is positioned in a vessel 1660. Transducers 1604 on the device 1661 are able to direct an energy wave to stimulate a target nerve outside of the vessel. The transducers and neural target may be in the same plane, or may be in different planes as illustrated in FIG. 16C. The vessel can be chosen to be adjacent to the target nerve. For example, some embodiments use an intravascular device in an internal jugular vein (IJV) to transvascularly stimulate a vagus nerve, or a specific neural pathway in the vagus nerve. The device can be implanted into other blood vessels too. The design is not limited to blood vessels, as similar designs can be used to position the device in other cavities or vessels that are not blood vessels.

Sonic intravascular device embodiments have a stent-like structure, with a shape-memory to fixate the device against the walls of the vessel without unacceptably obstructing blood flow in the vessel. Various shapes can be used for a stent-like structure, including helical shapes, cylindrical shapes, oval shapes and C-shapes. Some intravascular device embodiments are tethered to a controller via an intravascularly-fed lead, and some intravascular device embodiments are satellite devices. A satellite device is capable of operating autonomously or in a coordinated fashion with other satellites or a planet controller. Power and/or communication can be delivered via a wireless connection, such as an ultrasound or radiofrequency connection. Some intravascular device embodiments include one or more transducers positioned in a vessel or cavity, and are adapted to cooperate with other transducers to stimulate a target nerve. These other transducers can be positioned in the same vessel or cavity, another vessel or cavity, external to the body, on a nerve cuff, or otherwise positioned to deliver an energy wave toward the target nerve.

### External Transducers

Various embodiments use external transducers to selectively stimulate a nerve with constructive interference from energy waves from the external transducers. As those of ordinary skill in the art will understand upon reading and comprehending this disclosure, a number of external placement devices, such as bracelets, belts or collars.

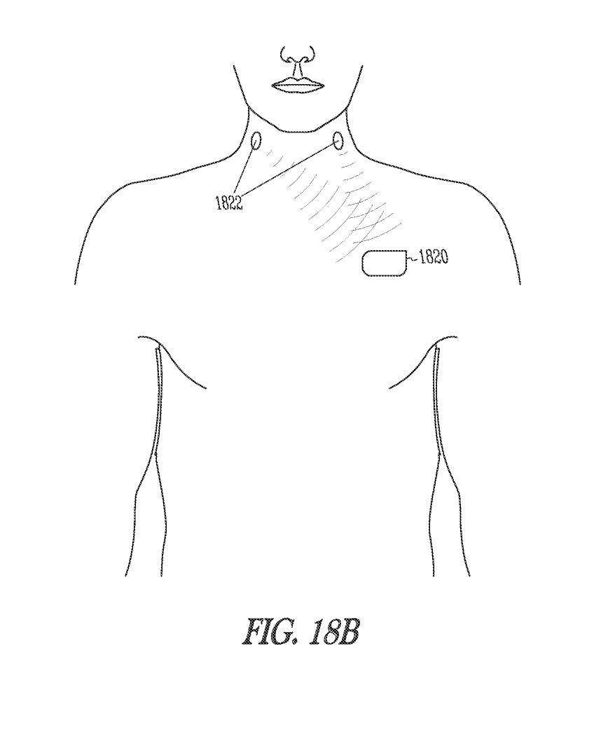

FIG. 17 illustrates an embodiment with external transducers. The figure illustrates a collar 1717 and right and left vagus nerves 1718A and 1718B. Transducers within the collar are capable of selectively stimulating neural pathways within the neck. The vagus nerve innervates a number of organs. Thus, specific vagal pathways, for example, can be selectively stimulated to achieve a desired therapy.

### Device Embodiments

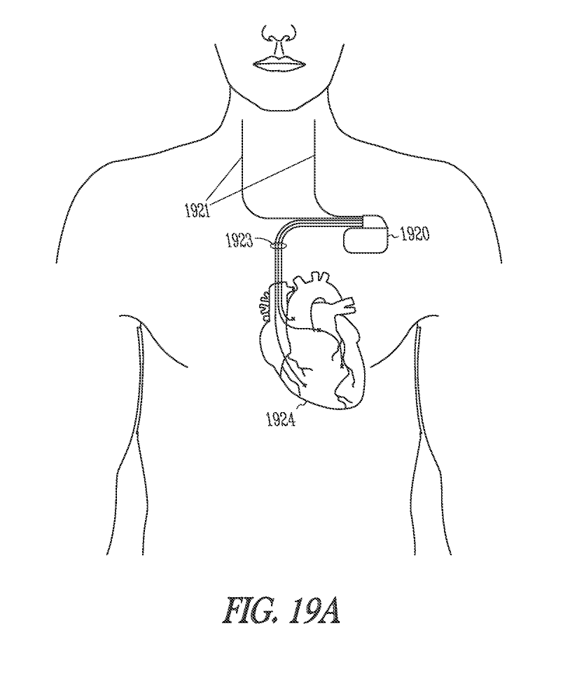

FIGS. 18A-18B illustrate some device embodiments that provide selective nerve stimulation. With reference to the illustrated embodiment in FIG. 18A, the IMD 1820 includes ports for connecting lead(s) 1821. Two leads are illustrated. Some embodiments use only one lead to stimulate neural target(s). The lead(s) 1821 include transducers adapted to provide the appropriate stimulation vectors for the neural target(s). An example of a neural target includes a vagus nerve. The present subject matter is not limited to a particular nerve to be stimulated. The IMD includes circuitry to control the generation and delivery of the electrical stimulation to the transducers on the lead(s). Some embodiments use subcutaneously-fed leads to position the transducers proximate to the neural target, using a nerve cuff, for example. Some embodiments use intravascularly-fed leads to position transducers within a vessel adjacent to a neural target to transvascularly stimulate the neural target(s). FIG. 18B illustrates a neural stimulation embodiment in a planet-satellite configuration. The IMD 1820 functions as a planet, and the transducers 1822 function as satellites wirelessly linked to the planet. Power and data can be sent over the wireless link using, for example, radio frequency or ultrasound technology.

Examples of satellite transducers include subcutaneous transducers, nerve cuff transducers and intravascular transducers.

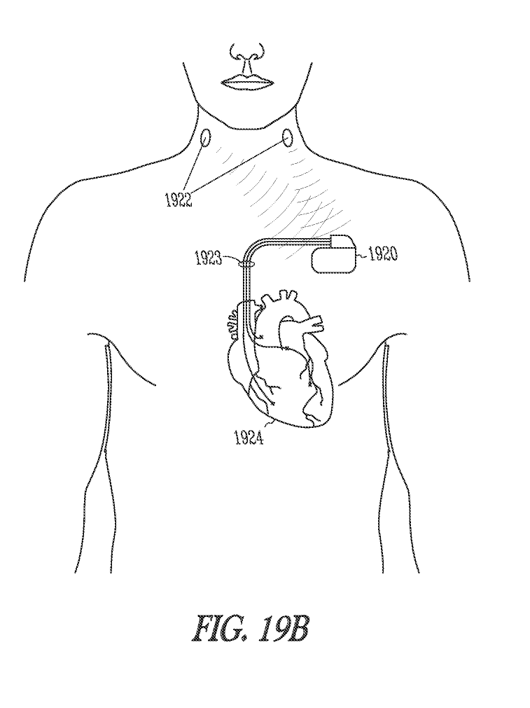

FIGS. 19A-19B illustrate some device embodiments that provide selective nerve stimulation and CRM therapy. FIG, 19A illustrates an IMD 1920 placed subcutaneously or submuscularly in a patient's chest with lead(s) 1923 positioned to provide a CRM therapy to a heart 1924, and with lead(s) 1921 positioned to stimulate a vagus nerve, by way of example and not by way of limitation. According to various embodiments, the leads 1923 are positioned in or proximate to the heart to provide a desired cardiac pacing therapy. In some embodiments, the lead(s) 1923 are positioned in or proximate to the heart to provide a desired defibrillation therapy. In some embodiments, the lead(s) 1923 are positioned in or proximate to the heart to provide a desired CRT therapy. Some embodiments place the leads in positions with respect to the heart that enable the lead(s) to deliver the combinations of at least two of the pacing, defibrillation and CRT therapies. According to various embodiments, neural stimulation lead(s) 1921 are subcutaneously tunneled to a neural target, and can have a nerve cuff electrode to stimulate the neural target. Some lead embodiments are intravascularly fed into a vessel proximate to the neural target, and use transducer(s) within the vessel to transvascularly stimulate the neural target. For example, some embodiments stimulate the vagus using electrode(s) positioned within the internal jugular vein.

FIG. 19B illustrates an implantable medical device (IMD) 1920 with lead(s) 1923 positioned to provide a CRM therapy to a heart 1924, and with satellite transducers 1922 positioned to stimulate at least one neural target as part of a therapy. The satellite transducers are connected to the IMD, which functions as the planet for the satellites, via a wireless link. Stimulation and communication can be performed through the wireless Examples of wireless links include RF links and ultrasound links. Although not illustrated, some embodiments perform myocardial stimulation using wireless links. Examples of satellite transducers include subcutaneous transducers, nerve cuff transducers and intravascular transducers.

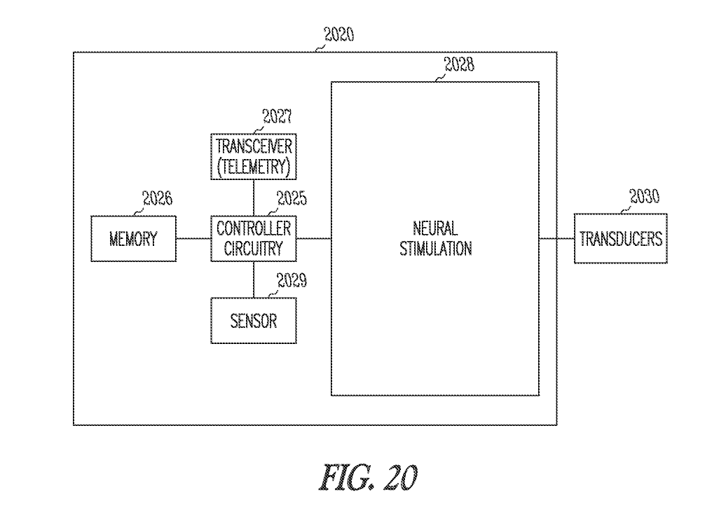

FIG. 20 illustrates an implantable medical device (IMD) 2020, according to various embodiments of the present subject matter. The illustrated IMD 2020 provides neural stimulation signals through transducers for delivery to predetermined neural targets. The illustrated device 2020 includes controller circuitry 2025 and memory 2026. The controller circuitry 2025 is capable of being implemented using hardware, software, and combinations of hardware and software. For example, according to various embodiments, the controller circuitry 2025 includes a processor to perform instructions embedded in the memory 2026 to perform functions associated with the neural stimulation therapy. For example, the illustrated device 2020 further includes a transceiver 2027 and associated circuitry for use to communicate with a programmer or another external or internal device. Various embodiments have wireless communication capabilities. For example, some transceiver embodiments use a telemetry coil to wirelessly communicate with a programmer or another external or internal device.

The illustrated device 2020 further includes neural stimulation circuitry 2028. Various embodiments of the device 2020 also includes sensor circuitry 2029. According to some embodiments, one or more leads are able to be connected to the sensor circuitry 2029 and neural stimulation circuitry 2028. Some embodiments use wireless connections between the sensor(s) and sensor circuitry, and some embodiments use wireless connections between the stimulator circuitry and transducers 2030. The neural stimulation circuitry 2028 is used to apply electrical stimulation pulses to transducers 2030 to provide desired neural stimulation to desired neural targets. In various embodiments, the sensor circuitry is used to detect and process nerve activity for use in determining when a desired neural target is being stimulated. In various embodiments, the sensor circuitry is used to detect and process surrogate parameters such as blood pressure, respiration, muscle tone, movement and the like, for use in determining when a desired neural target is being stimulated.

According to various embodiments, the stimulation circuitry 2028 includes modules to set or adjust any one or any combination of two or more of the following pulse features delivered to the transducers: the amplitude of the stimulation pulse, the frequency of the stimulation pulse, the burst frequency of the pulse, the wave morphology of the pulse, and the pulse width. The illustrated burst frequency pulse feature includes burst duration and duty cycle, which can be adjusted as part of a burst frequency pulse feature or can be adjusted separately. For example, a burst frequency can refer to the number of bursts per minute. Each of these bursts has a burst duration (an amount of time bursts of stimulation are provided) and a duty cycle (a ratio of time where stimulation is provided to total time). Thus, by way of example and not limitation, six bursts can be delivered during a one minute stimulation time (burst duration), where the length (pulse width) of each burst is five seconds and the time period between bursts is five seconds. In this example, the burst frequency is six bursts per minute, the stimulation time or burst duration is 60 seconds, and the duty cycle is 50% ((6 bursts×5 sec./burst)/60 seconds). Additionally, the duration of one or more bursts can be adjusted without reference to any steady burst frequency. For example, a single stimulation burst of a predetermined burst duration or a pattern of bursts of predetermined pulse width(s) and burst timing can be provided in response to a sensed signal. Furthermore, the duty cycle can be adjusted by adjusting the number of bursts and/or adjusting the duration of one or more bursts, without requiring the bursts to be delivered with a steady burst frequency. Examples of wave morphology include a square wave, triangle wave, sinusoidal wave, and waves with desired harmonic components to mimic white noise such as is indicative of naturally-occurring baroreflex stimulation. Additionally, various controller embodiments are capable of controlling a duration of the stimulation.

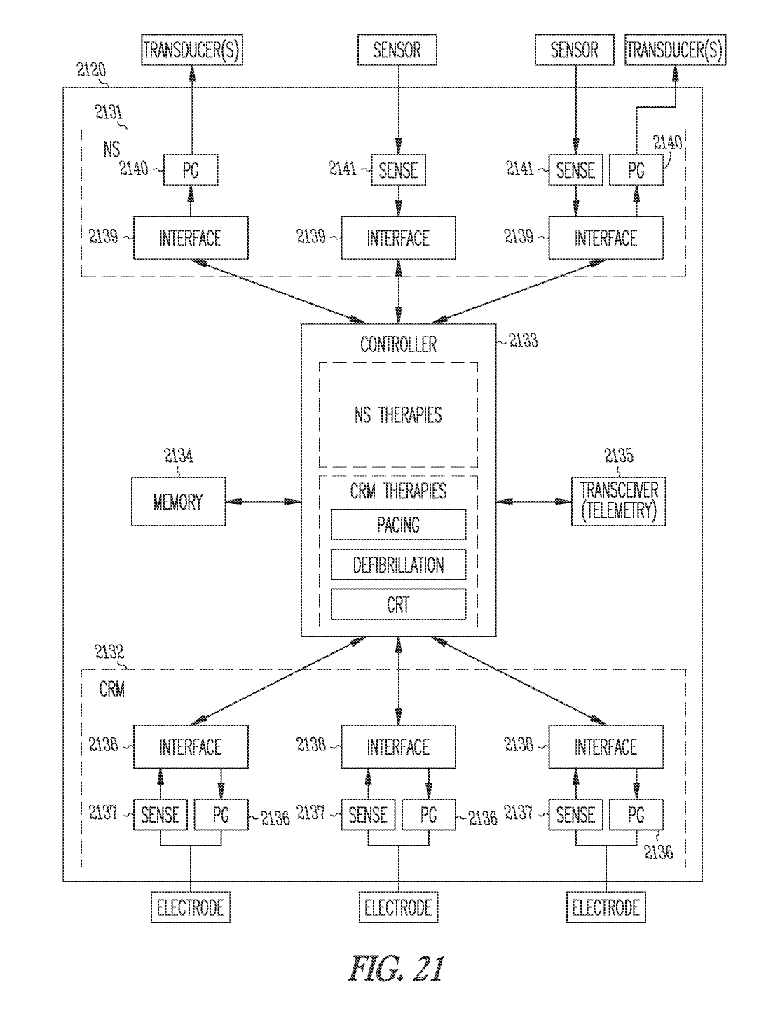

FIG. 21 illustrates an implantable medical device (IMD) 2120 having a neural stimulation (NS) component 2131 and cardiac rhythm management (CRM) component 2132, according to various embodiments of the present subject matter. The illustrated device includes a controller 2133 and memory 2134. According to various embodiments, the controller includes hardware, software, or a combination of hardware and software to perform the neural stimulation and CRM functions. For example, the programmed therapy applications discussed in this disclosure are capable of being stored as computer-readable instructions embodied in memory and executed by a processor. According to various embodiments, the controller includes a processor to execute instructions embedded in memory to perform the neural stimulation and CRM functions. Examples of CRM functions include bradycardia pacing, antitachycardia therapies such as antitachycardia pacing and defibrillation, and CRT (RCT). The illustrated device further includes a transceiver 2135 and associated circuitry for use to communicate with a programmer or another external or internal device. Various embodiments include a telemetry coil.

The CRM therapy section 2132 includes components, under the control of the controller, to stimulate a heart and/or sense cardiac signals using one or more electrodes. The CRM therapy section includes a pulse generator 2136 for use to provide an electrical signal through an electrode to stimulate a heart, and further includes sense circuitry 2137 to detect and process sensed cardiac signals. An interface 2138 is generally illustrated for use to communicate between the controller 2133 and the pulse generator 2136 and sense circuitry 2137. Three electrodes are illustrated as an example for use to provide CRM therapy. However, the present subject matter is not limited to a particular number of electrode sites. Each electrode may include its own pulse generator and sense circuitry. However, the present subject matter is not so limited. The pulse generating and sensing functions can be multiplexed to function with multiple electrodes.

The NS therapy section 2131 includes components, under the control of the controller, to stimulate a neural stimulation target and/or sense parameters associated with nerve activity or surrogates of nerve activity such as blood pressure and respiration. Three interfaces 2139 are illustrated for use to provide neural stimulation. However, the present subject matter is not limited to a particular number interfaces, or to any particular stimulating or sensing functions. Pulse generators 2140 are used to provide electrical pulses to transducer or transducers for use to stimulate a neural stimulation target. According to various embodiments, the pulse generator includes circuitry to set, and in some embodiments change, the amplitude of the stimulation pulse, the frequency of the stimulation pulse, the burst frequency of the pulse, and the morphology of the pulse such as a square wave, triangle wave, sinusoidal wave, and waves with desired harmonic components to mimic white noise or other signals. Sense circuits 2141 are used to detect and process signals from a sensor, such as a sensor of nerve activity, blood pressure, respiration, and the like. The interfaces 2139 are generally illustrated for use to communicate between the controller 2133 and the pulse generator 2140 and sense circuitry 2141. Each interface, for example, may be used to control a separate lead. Various embodiments of the NS therapy section only include a pulse generator to stimulate neural targets such a vagus nerve.

### Advanced Patient Management

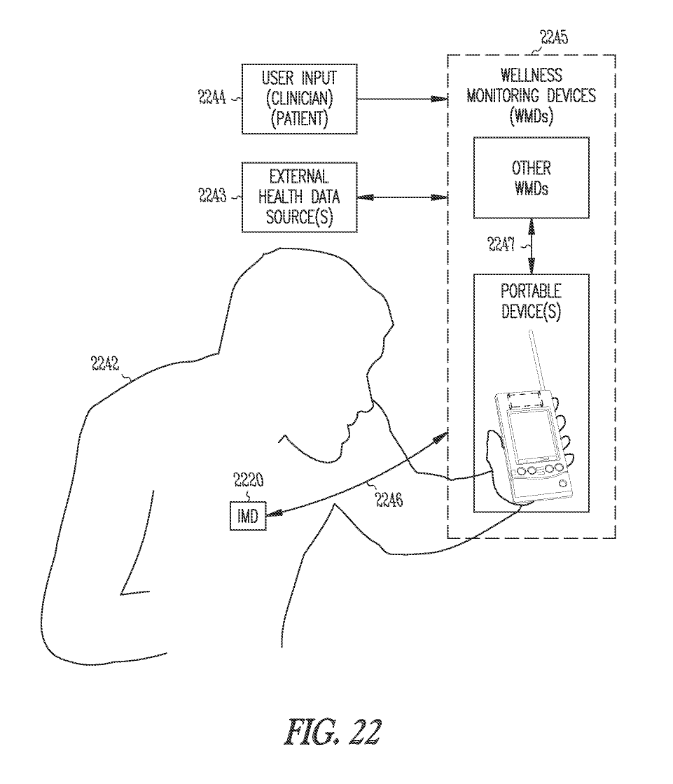

Various embodiments of the present subject matter use the neural stimulation device as an IMD within an advanced patient management (APM) system. FIG. 22 illustrates an ARM system according to various embodiments of the present subject matter. A patient 2242 is illustrated with an implantable medical device (IMD) 2220. Generally, the 1MD includes one or more IMDs that provide internal therapy and/or acquire or sense internal data parameters. In various embodiments, the IMD is a neural stimulation device. In some embodiments, the IMD also functions as a CRM device that provides CRM stimulation and also senses one or more physiological parameters of a heart. Other IMDs that sense parameters and/or provide therapy, including various electrical and drug therapy, are within the scope of the present subject matter.

In various embodiments, at least one 1MD 2220 provides internal data such as heart rhythm, breathing, activity, and stimulation parameters, and timing. In various embodiments. IMD-provided data includes parameters sensed by the IMD and/or parameters provided by interrogating the IMD to obtain device performance status. The illustrated system also includes one or more external data source(s) 2243 that provide health-related parameters. The external health-related parameters supplement the internal parameters and/or provide a diagnostic context to the internal health-related parameters. Examples of external source(s) of health data include: external sensing devices such as body temperature thermometers, blood pressure monitors, and the like; room temperature thermometers, light sensors and the like; databases such as patient history databases that are found hospitals or clinics and that may include information such as medical test results and family history; a web server database (a database accessible through a global communication network—e.g. Internet) that may include information regarding environment, medication interaction, and the like; databases and/or user inputs regarding mental/emotional and diet parameter types; and other external data sources capable of providing health-related parameters.

The illustrated system also includes a user input 2244 through which a user is able to input additional health-related parameters for use by a wellness monitoring device (WMD) 2245. In various embodiments, the user input includes a touch screen on a PDA or other device, a keyboard and mouse on a computer, and the like. In various embodiments, a patient is able to input additional health-related parameters for use by the wellness monitoring device. In various embodiments, a clinician is able to input additional health-related parameters for use by the WMD. The WMD 2245 is illustrated by dotted line, and includes one or more devices. In various embodiments, the at least one IMD communicates wirelessly with at least one WMD, as shown by communication link 2246. In various embodiments that include multiple WMDs, the WMDs are able to communicate with each other, as shown via communication link 2247. In various embodiments, the WMD(s) includes portable devices that are external to the body of patient such as a PDA, (variously referred to as a personal digital, or data, assistant), a portable telephone (including a cellular telephone or a cordless telephone), a pager (one way or two way), a handheld, palm-top, laptop, portable or notebook computer, or other such battery operated portable communication device. In various embodiments, the WMD(s) includes programmers. In various embodiments, the WMD(s) includes various non-portable devices such as larger computers or computer enterprise systems. In various embodiments of the present subject matter, the WMD (which includes one or more devices) includes a display on which parameter trends are capable of being displayed. Some WMD embodiments provide analysis of internal and external (both voluntary and involuntary) parameters. In various embodiments, the WMD includes computer and programming that conducts data analysis suitable for use in managing patient health and medical care.

### Method to Selectively Stimulate Desired Neural Target

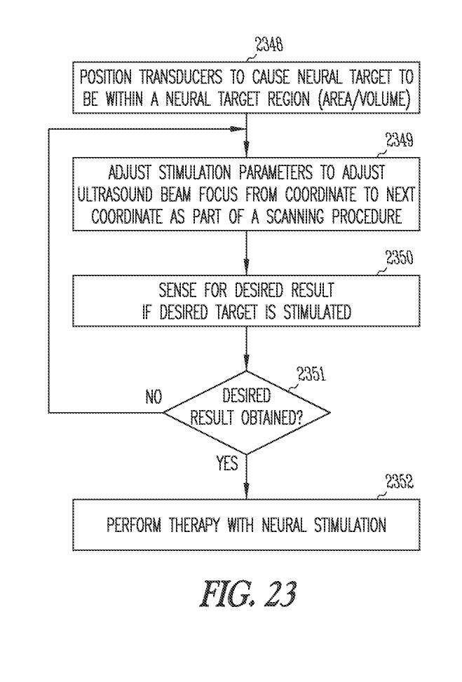

FIG. 23 illustrates a method to selectively stimulate a desired neural target, according to various embodiments. At 2348, transducers are positioned to cause a neural target to be stimulated to be within a neural target region. At 2349, stimulation parameters are adjusted to focus an energy beam (e.g. ultrasound beam) from the transducers to constructively interfere from coordinate to coordinate as part of a scanning procedure. At 2350, a desired result is sensed or otherwise detected to indicate that the desired neural target has been stimulated. If the desired result has not been obtained at 2351, the process proceeds to 2349 to adjust the focus to another coordinate within the scanning procedure. If the desired result has been obtained at 2351, the process proceeds to 2352 to perform a therapy with the neural stimulation of the desired neural target.

One of ordinary skill in the art will understand that, the modules and other circuitry shown and described herein can be implemented using software, hardware, and combinations of software and hardware. As such, the illustrated modules and circuitry are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined.

In various embodiments, the methods provided above are implemented as a computer data signal embodied in a carrier wave or propagated signal, that represents a sequence of instructions which, when executed by a processor cause the processor to perform the respective method. In various embodiments, methods provided above are implemented as a set of instructions contained on a computer-accessible medium capable of directing a processor to perform the respective method. In various embodiments, the medium is a magnetic medium, an electronic medium, or an optical medium.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments as well as combinations of portions of the above embodiments in other embodiments will be apparent to those of skill in the art upon reviewing the above description. The scope of the present subject matter should be determined with reference to the appended claims, along with the full scope of equivalents to which such claims are entitled.

1. (canceled)

2. A method for providing selective stimulation of only some neural pathways within a neural target region, comprising:
performing a scanning procedure to test selective stimulation at a sequence of coordinates within the neural target region to identify when targeted neural pathways in the neural target region are being selectively stimulated, wherein performing the scanning procedure includes sequentially delivering an ultrasound beam focus to individual ones of the coordinates within the sequence of coordinates and sensing for a desired physiological response, wherein:
sequentially delivering the ultrasound beam focus includes generating at least a first ultrasound energy wave with a first amplitude from a first position and a second ultrasound energy wave with a second amplitude from a second position to sequentially provide constructive interference at the individual ones of the coordinates, wherein the first amplitude alone is not sufficient to stimulate the neural target region, the second amplitude alone is not sufficient to stimulate the neural target region, and the constructive interference is sufficient to stimulate the individual ones of the coordinates the neural target region; and
the desired physiological response indicates when the ultrasound beam focus is stimulating the targeted neural pathways;
identifying a target coordinate from the tested sequence of coordinates to be used for selective neural stimulation of the targeted neural pathways, wherein the desired physiological response was sensed when the target coordinate was stimulated by the ultrasound beam focus; and
delivering a therapy including delivering the ultrasound beam focus to the target coordinate to selectively stimulate the targeted neural pathways.

3. The method of claim 2, wherein the sequence of coordinates within the neural target region is a sequence of two-dimensional coordinates.

4. The method of claim 2, wherein the sequence of coordinates within the neural target region is a sequence of three-dimensional coordinates.

5. The method of claim 2, wherein sequentially delivering an ultrasound beam focus to individual ones of the coordinates within the sequence of coordinates includes adjusting at least one phase of the ultrasound energy waves to move the ultrasound beam focus to the individual ones of the coordinates within the sequence of coordinates.

6. The method of claim 2, wherein the neural target region is a nerve trunk with a plurality of axons, and only some of the plurality of axons are stimulated by the selective stimulation.

7. The method of claim 2, wherein the neural target region is a vagus nerve with a plurality of axons, and only some of the plurality of axons are stimulated by the selective stimulation.

8. The method of claim 2, wherein the first position and the second position are positions on a nerve cuff.

9. The method of claim 2, wherein the first position and the second position are intravascular positions.

10. The method of claim 2, wherein sensing for the desired physiological response includes sensing nerve activity.

11. The method of claim 2, wherein sensing for the desired response includes sensing blood pressure, or sensing respiration, sensing muscle tone, sensing movement or sensing cardiac activity.

12. A system for selectively stimulating on some neural pathways within a neural target region, comprising:
a plurality of ultrasound transducers, each ultrasound transducer being adapted to be positioned to direct an ultrasound signal toward individual coordinates in the neural target region;
a sensor configured to sense for a desired response when targeted neural pathways within the neural target region are stimulated; and
a processor adapted to:
perform a scanning procedure to test selective stimulation at a sequence of the individual coordinates within the neural target region to identify when targeted neural pathways in the neural target region are being selectively stimulated, wherein performing the scanning procedure includes:
sequentially deliver an ultrasound beam focus to individual ones of the coordinates within the sequence of coordinates and sensing for a desired physiological response, including generate at least a first ultrasound energy wave with a first amplitude from a first position and a second ultrasound energy wave with a second amplitude from a second position to sequentially provide constructive interference at the individual ones of the coordinates, wherein the first amplitude alone is not sufficient to stimulate the neural target region, the second amplitude alone is not sufficient to stimulate the neural target region, and the constructive interference is sufficient to stimulate the individual ones of the coordinates the neural target region, the desired physiological response indicating when the ultrasound beam focus is stimulating the targeted neural pathways;
identify a target coordinate from the tested sequence of coordinates to be used for selective neural stimulation of the targeted neural pathways, wherein the desired physiological response was sensed when the target coordinate was stimulated by the ultrasound beam focus;
deliver a therapy including delivering the ultrasound beam focus to the target coordinate to selectively stimulate the targeted neural pathways.

13. The system of claim 12, wherein the sequence of coordinates within the neural target region is a sequence of two-dimensional coordinates.

14. The system of claim 12, wherein the sequence of coordinates within the neural target region is a sequence of three-dimensional coordinates.

15. The system of claim 12, wherein the processor is configured to adjust at least one phase of the ultrasound energy waves to move the ultrasound beam focus to the individual ones of the coordinates within the sequence of coordinates.

16. The system of claim 12, wherein the neural target region is a nerve trunk with a plurality of axons, the system being configured to stimulate only some of the plurality of axons.

17. The system of claim 12, wherein the neural target region is a vagus nerve with a plurality of axons, the system being configured to stimulate only some of the plurality of axons.

18. The system of claim 12, further comprising a nerve cuff for placement around a targeted nerve, the plurality of transducers being located on the nerve cuff and configured for use to selectively stimulate some neural pathways within the targeted nerve.

19. The system of claim 12, further comprising an intravascular device for placement in vascular next to a targeted nerve, the plurality of transducers being located on the intravascular and configured for use to selectively stimulate some neural pathways within the targeted nerve.

20. The system of claim 12, wherein the sensor includes a nerve activity sensor.

21. The system of claim 12, wherein the sensor includes a blood pressure sensor, a respiration sensor, a muscle tone sensor, or a cardiac activity sensor.
